(19) United States
(12) Patent Application Publication
(10) Pub. No.: US 2022/0111348 A1
(43) Pub. Date: Apr. 14, 2022

(54) Machine Learning-Based Online Optimization Of Solid Phase Slug Flow Peptide Synthesis

(71) Applicant: MyTide Therapeutics, Inc., Boston, MA (US)

(72) Inventors: Kevin Shi, Boston, MA (US); Liudi Yang, Franklin, MA (US)

(21) Appl. No.: 17/535,210

(22) Filed: Nov. 24, 2021

Related U.S. Application Data
(63) Continuation-in-part of application No. PCT/US2020/037441, filed on Jun. 12, 2020.
(60) Provisional application No. 62/861,821, filed on Jun. 14, 2019.
(60) Provisional application No. 63/009,563, filed on Apr. 14, 2020.

(51) Int. Cl. B01J 19/00; C07K 1/04; G06N 20/00

(52) U.S. Cl. CPC B01J 19/0046; C07K 1/042; G06N 20/00; B01J 2219/00286; B01J 2219/00695; B01J 2219/0059; B01J 2219/00596; B01J 2219/00689; B01J 2219/00423

(57) ABSTRACT

The present disclosure provides computer-based methods and systems for controlling peptide synthesis. An embodiment begins by providing a manufacturing process that synthesizes peptides using solid phase slug flow. In turn, the manufacturing process is automated through use of a machine learning engine by selecting values for operating conditions for the manufacturing process. In such an embodiment, a given operating condition is flow rate profile. An embodiment generates an indication of the selected values for the operating conditions and controls the manufacturing process therewith.

### RELATED APPLICATIONS

This application is a continuation-in-part of International Application No. PCT/US2020/037441, which designated the United States and was filed on Jun. 12, 2020, published in English, which claims the benefit of U.S. Provisional Application No. 62/861,821, filed on Jun. 14, 2019 and claims the benefit of U.S. Provisional Application No. 63/009,563 filed on Apr. 14, 2020.

The entire teachings of the above applications are incorporated herein by reference.

### GOVERNMENT SUPPORT

This invention was made with government support under Grant No. 1938756 from National Science Foundation. The government has certain rights in the invention.

### TECHNICAL FIELD

The present disclosure relates to control of semi-continuous or continuous manufacturing techniques for rapid production of peptide and protein production using solid phase peptide synthesis (SPPS). In particular, embodiments provide functionality to control manufacturing processes that synthesize peptides using solid phase slug flow.

### BACKGROUND

Peptides and proteins play a vital role by mediating an extensive range of biological processes, acting as signaling molecules, antibiotics, or hormones. Due to the highly specific interaction with their biological targets, peptides and proteins have been widely used in medicine and represent a growing subset of the therapeutic market.

### SUMMARY

With the increased use of peptides and proteins, functionality is needed to improve the control of peptide and protein production methods. Embodiments provide such functionality.

An embodiment uses predictive models to improve peptide synthesis by predicting synthesizability under a variety of possible simulated conditions prior to synthesis. The conditions with the highest probability of success/most favorable results are selected, and then, that selected protocol is performed to generate the peptide. After performing the synthesis, measured results are recorded, the prediction quality is evaluated, and the results are added to a running dataset to inform future predictions. For small numbers of model evaluations, this functionality can be implemented using local processing and for larger scale predictions, this functionality can be implemented on graphics processing units (GPUs) and/or cloud resources as a microservice. The synthesizability metrics can correspond to the end-to-end model outputs.

An example embodiment is directed to a computer-based method for controlling peptide production that begins by providing a manufacturing process that synthesizes peptides using solid phase slug flow. The method automates the provided manufacturing process through use of a machine learning engine by selecting values for operating conditions for the manufacturing process. In turn, an indication of the selected values for the operating conditions is generated. According to an embodiment, a given operating condition is flow rate profile.

Another embodiment selects values for operating conditions for the manufacturing process by determining candidate values for the operating conditions for a plurality of peptide production scenarios. With these determined candidate values, quality of each of the plurality of peptide production scenarios is predicted by using the determined candidate values in the machine learning engine. The machine learning engine is configured to output an indication of predicted production quality given candidate values for the operating conditions. In turn, a peptide production scenario from among the plurality of peptide production scenarios is selected based upon the indication of predicted production quality for each of the plurality of peptide production scenarios. In such an embodiment, the candidate values for the operating conditions of the selected peptide production scenario are the selected values for the operating conditions for the manufacturing process.

Embodiments can employ any number of methods to determine the candidate values for the operating conditions. One such embodiment randomly generates candidate values between an upper bound and a lower bound for an operating condition. Another embodiment generates candidate values in set increments between an upper bound and a lower bound for an operating condition.

According to an embodiment, the indication of predicted production quality indicates at least one of: production yield, peptide purity, and production time. In an example embodiment, the indication of production yield corresponds to an integral of ultraviolet (UV) absorbance trace over time of flow-through reaction products. In an embodiment, the indication of production yield corresponds to an extent of reaction determined by dividing a measured UV trace by an instantaneous flow rate and multiplying by a constant. According to an example embodiment, the output of the machine learning engine corresponds to a quantity that can be calculated from operating the peptide manufacturing system (extent of reaction, time taken, etc.). For instance, where the output of the machine learning engine is an indication of production yield, this machine learning output may be the same value, in the example of extent of reaction, that is determined by operating the system (peptide manufacturing system), measuring a UV trace, and dividing the measured UV trace by instantaneous flow rate and multiplying by a constant.

As noted above, in an embodiment, an operating condition includes flow rate profile. Flow rate profile may indicate flow rates for each of a plurality of stages of the peptide manufacturing process. For instance, the flow rate profile may include flow rates for load, couple, capping, deprotect, and wash stages of the provided peptide manufacturing process.

In addition to including flow rate profile, in embodiments, the operating conditions may also include at least one of: current amino acid position, current amino acid identity, previous amino acid, physical properties of an amino acid, chemical properties of an amino acid, oscillation frequency, and temperature. In an embodiment, temperature indicates a temperature for each of a plurality of stages of the manufacturing process, e.g., the load, couple, capping, deprotect, and wash stages of the provided peptide manufacturing process.

Yet another embodiment controls the manufacturing process in accordance with the generated indication of the selected values for the operating conditions. Further, according to an embodiment, the generated indication of the selected values for the operating conditions enables computer automated control of the manufacturing process.

Another embodiment is directed to a system for controlling a manufacturing process that synthesizes peptides using solid phase slug flow. In one such embodiment, the system includes a processor and a memory with computer code instructions stored thereon. The processor and the memory, with the computer code instructions, are configured to cause the system to implement any embodiments or combination of embodiments described herein.

Yet another embodiment is directed to a computer program product for controlling a manufacturing process that synthesizes peptides using solid phase slug flow. The computer program product comprises one or more non-transitory computer-readable storage devices and program instructions stored on at least one of the one or more storage devices. When the program instructions are loaded and executed by a processor, the program instructions cause an apparatus associated with the processor to implement any embodiments or combination of embodiments described herein.

It is noted that embodiments of the method, system, and computer program product may be configured to implement any embodiments or combination of embodiments described herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows an illustration of different slugs of material for performing peptide couplings that may be controlled using embodiments.

FIG. 2 is a flowchart of a method for controlling peptide production according to an embodiment.

FIG. 3 shows an image of a robotic flow chemistry system for performing peptide synthesis that may be operated using embodiments.

FIGS. 4 and 5 show illustrations of solid phase slug flow (SPSF) synthesis platforms that may be controlled using embodiments.

FIG. 6 is a graphical depiction of an embodiment that uses machine learning to synthesize a peptide.

FIG. 7 is a flowchart of a method for training a machine learning model and controlling peptide production using the trained machine learning model according to an embodiment.

FIG. 8A is a plot showing baseline input for a method of peptide production.

FIG. 8B is a plot of an output characteristic of a method of peptide production implemented using the input shown in FIG. 8A.

FIG. 9A is a plot showing the input from FIG. 8A optimized using an embodiment.

FIG. 9B is a plot of an output characteristic of a method of peptide production implemented using the input shown in FIG. 9A.

FIG. 10 depicts a computer network or similar digital processing environment in which embodiments of the present invention may be implemented.

FIG. 11 is a diagram of an example internal structure of a computer in the environment of FIG. 10.

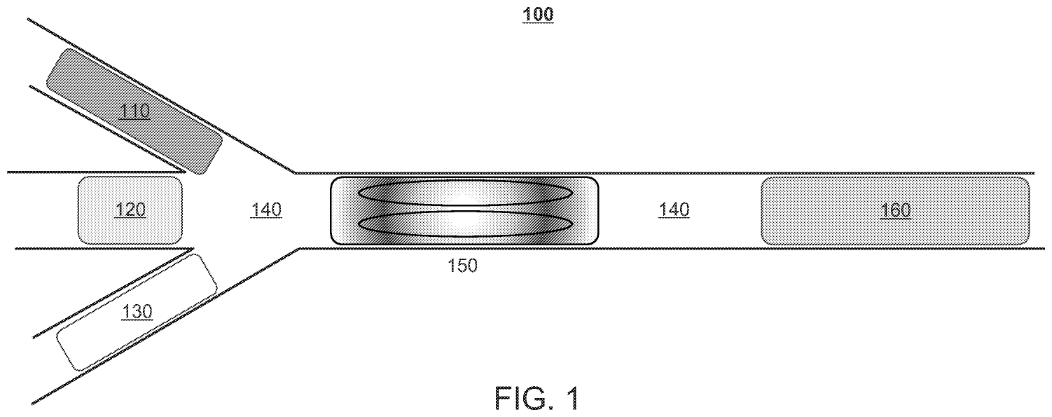

### DETAILED DESCRIPTION

A description of example embodiments follows.

### Definitions

For purposes of the present disclosure, the following definitions will be used unless expressly stated otherwise:

The terms “a”, “an”, “the” and similar referents used in the context of describing the present disclosure are to be construed to cover both the singular and the plural, unless otherwise indicated herein or clearly contradicted by context. All methods described herein, can be performed in any suitable order unless otherwise indicated herein or otherwise clearly contradicted by context. The use of any and all examples, or exemplary language (e.g., “such as”) provided herein, is intended merely to better illuminate the present disclosure and does not pose a limitation on the scope of the disclosure otherwise claimed. No language in the present specification should be construed as indicating any unclaimed element is essential to the practice of the disclosure.

The term “about” in relation to a given numerical value, such as for temperature and period of time, is meant to include numerical values within 10% of the specified value.

As used herein, except where the context requires otherwise, the term “comprise” and variations of the term, such as “comprising”, “comprises” and “comprised”, are not intended to exclude further additives, components, integers or steps. The terms “including” and “comprising” may be used interchangeably. As used herein, the phrases “selected from the group consisting of”, “chosen from”, and the like, include mixtures of the specified materials. Where a numerical limit or range is stated herein, the endpoints are included. Also, all values and subranges within a numerical limit or range are specifically included as if explicitly written herein. References to an element in the singular are not intended to mean “one and only one” unless specifically stated, but rather “one or more”. Unless specifically stated otherwise, terms such as “some” refer to one or more, and singular terms such as “a”, “an” and “the” refer to one or more.

The term “substantially” as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

It is understood that the specific order or hierarchy of steps in the methods or processes disclosed is an illustration of exemplary approaches. Based upon design preferences, it is understood that the specific order or hierarchy of steps in the methods or processes may be rearranged. Some of the steps may be performed simultaneously. The accompanying methods claims present elements of the various steps in a sample order, and are not meant to be limited to a specific hierarchy or order presented. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice-versa.

It will be readily understood that the aspects and embodiments, as generally described herein, are exemplary. The following more detailed description of various aspects and embodiments are not intended to limit the scope of the present disclosure, but is merely representative of various aspects and embodiments. Moreover, the methods and systems disclosed herein may be changed by those skilled in the art without departing from the scope of the present disclosure. Unless defined otherwise, all technical and scientific terms used herein have the same meaning as is commonly understood by one of skill in the art to which this disclosure belongs. All publications and patents referred to herein are incorporated by reference.

Embodiments relate to control of semi-continuous or continuous manufacturing techniques for rapid production of peptide and protein production using solid phase peptide synthesis (SPPS). In particular, embodiments provide functionality to control a manufacturing process that synthesizes peptides using solid phase slug flow.

SPPS has become the predominant manufacturing methodology for small and large quantities of peptides. Solid-phase peptide synthesis is a process in which amino acids or peptides are added to amino acids, peptides or proteins that are immobilized on a solid support, e.g., resin. This technique utilizes standard batch chemistry or semi-batch methodologies to grow a peptide chain on a resin support. Growth of the peptide chain is achieved through repeated cycles of coupling where a deprotection step exposes an active site of the amino acid, peptide, or protein that are immobilized on the resin, and a coupling step forms new amide bonds at the exposed active sites.

An example of SPPS is shown in the system 100 where the liquid slugs containing amino acid 110, base 120, and activator 130 are flowed through a channel and are separated by separation media 140. The amino acid 110, base 120, and activator 130 are trapped at capture zone 150 where activation of amino acid 110 occurs by mixing. Capture zone 150 is further separated by separation media 140 forming slug 160, which can be carried to an amino acid, peptide, or protein that is immobilized on a solid support resin. System 100 enables controlled delivery of reagents for the synthesis of peptides and proteins during SPPS.

Yield, purity, and efficiency in SPPS are highly dependent on the ability to consistently deliver the required plurality of reagents to each particular active site on the amino acids, peptides, or proteins that are immobilized on the resin support, and then completely remove excess reagents and reaction by-products from the resin support before introducing the plurality of reagents for subsequent steps. Methods exists that attempt to optimize yield, purity, and efficiency (amongst other quality metrics) of such peptide synthesis processes, but these existing optimization methods are inadequate.

Existing processes for optimizing peptide synthesis typically rely on hardcoding previously established experimental conditions and/or performing design of experiments to explore a set of process conditions as a one-off process. The existing optimization methods happen before (prior to) production synthesis runs. Existing processes and technologies also typically use fixed sets of process conditions, either for all amino acids or by amino acid type, without regard for the growing amino acid chain. As such, the existing processes for peptide synthesis optimization rely on fixed conditions determined in separate experiments. Existing synthesizers are also slower for each amino acid coupling. The existing synthesizers use very different operating conditions, e.g., reagent concentrations, continuous addition of reactants, continuous removal of products, mixing strategy, and preheating strategy, amongst others. There exist fully continuous flow reactors that do not (and cannot) use the start/stop/different flow rate profiles utilized by embodiments. There also exist batch reactors that do not add/remove material during the course of the reaction. As such, optimal conditions for getting good synthesis results, e.g., yield and purity, amongst others, are different for the existing systems in comparison to embodiments.

Embodiments solve these problems by using a machine learning engine to control peptide synthesis. Embodiments can determine conditions to optimize (maximize produced product) given some fixed time or minimize elapsed time or production time given some target amount of product yield of synthesis of a solid phase peptide manufacturing method. Moreover, embodiments can determine conditions to operate solid phase slug flow synthesizers, such as those described in the International Application No. PCT/US2020/037441, published as WO 2020/252266, the contents of which are herein incorporated by reference.

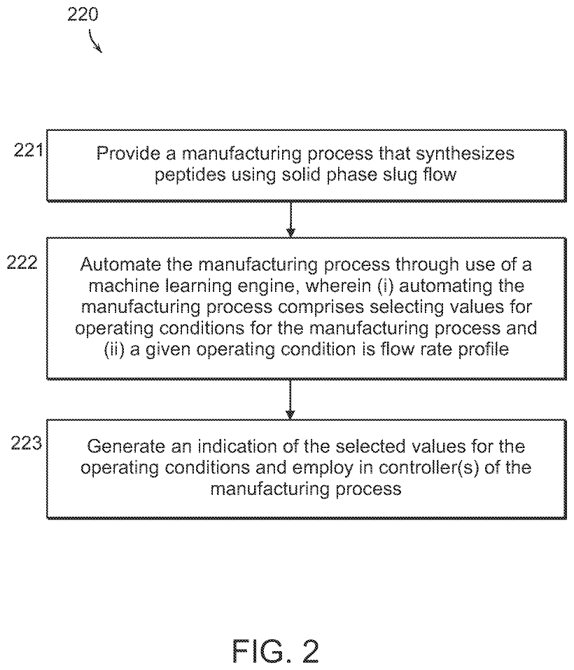

FIG. 2 is a flowchart of a method 220 for controlling peptide production according to an embodiment. The method 220 begins at step 221 with providing a subject manufacturing process that synthesizes peptides using solid phase slug flow. At step 221, any such slug flow peptide manufacturing process that is known in the art may be provided. Further, in an embodiment of the method 220, the subject manufacturing process provided at step 221 is any such solid phase slug flow process described in International Application No. PCT/US2020/037441. An embodiment of the method 220 is computer implemented. In such an embodiment, the subject manufacturing process is provided at step 221 by interfacing with, or otherwise communicatively coupling with a solid phase slug flow synthesis machine and/or machine controller. Further, in another computer implemented embodiment of the method 220, the computing device implementing the method 220 is integrated into the controls and data handling portions of a peptide manufacturing process/machine.

To continue, at step 222 the method 220 automates the provided manufacturing process through use of a machine learning engine by automatically selecting values for operating conditions for the subject manufacturing process. In such an embodiment, a given operating condition is flow rate profile.

An example embodiment of the method 220 selects values for the operating conditions for the subject manufacturing process at step 222 by first, determining candidate values for the operating conditions for a plurality of peptide production scenarios. Embodiments of the method 220 can employ a variety of techniques to determine the candidate values for the operating conditions. One such technique randomly generates candidate values between an upper bound and a lower bound for an operating condition. Another embodiment generates candidate values in set (certain, predefined) increments between an upper bound and a lower bound for an operating condition.

To illustrate functionality employed at step 222, consider an embodiment of the method 220 where the operating conditions are flow rate profile and temperature profile. In such an illustrative embodiment, there a three production scenarios, A, B, and C and respective flow rate profile values and temperature profile values are determined for each scenario, A, B, and C. In turn, quality of each of the plurality of peptide production scenarios (A, B, and C) is predicted by using the determined candidate values in the machine learning engine. In such an embodiment, the machine learning engine is configured to output an indication of predicted production quality given the candidate values for the operating conditions. As such, the embodiment inputs the candidate values for the operating conditions to the machine learning engine and the machine learning engine outputs an indication of predicted production quality for each peptide production scenario (A, B, and C).

To continue, a peptide production scenario (A, B, or C) from among the plurality of peptide production scenarios (A, B, and C) is selected based upon the indication of predicted production quality for each of the plurality of peptide production scenarios. For example, the peptide production scenario that best satisfies a desired outcome, e.g., yield, production time (elapsed time), etc., is selected. In such an embodiment, the candidate values for the operating conditions of the selected peptide production scenario correspond to the selected values for the operating conditions for the subject manufacturing process. In this way, output of step 222 (feeding to step 223) includes the selected values for the operating conditions of the subject manufacturing process.

According to an embodiment of the method 220, the indication of predicted production quality provided by the machine learning engine indicates at least one of: production yield, peptide purity, and production time (elapsed time). In an embodiment the indication of production yield corresponds to an integral of ultraviolet (UV) absorbance trace over time of flow-through reaction products. In another example embodiment, the indication of production yield corresponds to an extent of reaction that can be determined by dividing a measured UV trace by an instantaneous flow rate and multiplying by a constant. According to an embodiment, the output of the machine learning engine corresponds to a quantity that can be calculated from operating the peptide manufacturing system (extent of reaction, time taken, etc.). For instance, where the output of the machine learning engine is an indication of production yield, the machine learning output may be the same value (in the example of extent of reaction) that is determined by operating the system, measuring a UV trace, and dividing the measured UV trace by instantaneous flow rate and multiplying by a constant.

In an embodiment, the predicted indication of production yield for the synthesizer is in terms that can be calculated “online” directly during synthesis without having to wait for downstream processing. The integral of UV absorbance over time gives a measure of extent of deprotection, which is the extent of reaction, which is directly proportional to production yield. The integral of UV absorbance over time gives an imperfect measure of extent of deprotection because of changes in flow rate (the flow profile of slug flow). To illustrate, consider the case of zero flow rate. When there is zero flow rate there is nonzero UV absorbance for some time interval, giving a nonzero integral, but there is no material entering or leaving the detector (or reactor). The improved method utilized in embodiments normalizes the instantaneous UV absorbance by dividing by the instantaneous flow rate (and multiplying by some constant factor like a fixed nominal flow rate for convenience). Similarly, consider the case of infinite flow rate. The maximum concentration of reactants enters the reactor and the minimum concentration of products leaves the reactor. By Le Chatelier's principle, the rate of reaction is maximized, but the product is infinitely diluted so the instantaneous UV absorption is zero. Once again, the improved method utilized in embodiments provides a more accurate view of the performance characteristics of the reactor and flow system.

Returning to FIG. 2, at step 223, the method 220 generates an indication of the selected values for the operating conditions. The generated indication may be stored in computer or processor memory. Further, the generated indication may take any form that can be communicated. For instance, in an embodiment, the generated indication of the selected values for the operating conditions is in the form of controller or processor commands that enable control of the subject manufacturing process. An embodiment of the method 220 may also control the subject manufacturing process in accordance with the generated indication of the selected values for the operating conditions. For instance, in a computer implemented embodiment of the method 220, the computer device implementing the method 220 may communicate with the peptide manufacturing machine. The computer device implementing the method 220 communicates the selected values for the operating conditions to the peptide manufacturing machine causing the subject manufacturing machine to operate in accordance with the selected values.

The method 220 can control a peptide synthesizer. In one such implementation, the peptide synthesizer has a controller, and the controller combines a peptide specification (desired amino acid sequence), recipe template (a single amino acid's reactions), and parameters (flow rate profile, temperature, etc., determined through use of the method 220) to create an overall control scheme for synthesizing the entire peptide. According to an embodiment, this control scheme takes the form of a directed acyclic graph of control steps, where multiple edges leaving a node indicate concurrent execution and multiple edges joining at a node indicate waiting for all the attached preceding nodes to finish. An embodiment implements the controller as an interpreter that traverses this graph, executing the steps with the supplied parameters. Inside each step are instructions that actuate the pumps, valves, temperature controllers, etc. of the peptide synthesizer. The UV absorption, temperature, etc. are measured continuously and stored in computer working memory according to an embodiment. These values may then be used to refine the training of the machine learning model.

As noted above, in an embodiment of the method 220, an operating condition is flow rate profile. Flow rate profile may indicate flow rates for each of a plurality of stages of the peptide manufacturing process. For instance, the flow rate profile may include flow rates for load, couple, capping, deprotect, and wash stages of the subject peptide manufacturing process provided at step 221. In addition to including flow rate profile, in embodiments of the method 220, the operating conditions may also include at least one of: current amino acid position, current amino acid identity, previous amino acid, physical properties of an amino acid, chemical properties of an amino acid, oscillation frequency, and temperature. In an embodiment, temperature indicates a temperature for each of a plurality of stages of the manufacturing process.

Slug flow for solid phase peptide synthesis, e.g., solid phase slug flow (SPSF), enables integration for automation and robotics. Because SPSF can be integrated with automation controls and robotics, SPSF allows for precision reaction kinetics, excellent mixing control, efficient coupling transformations, and inline analytics for real-time monitoring of reactions and process steps. Embodiments of the present invention can be implemented in such automated and robotic peptide synthesis applications to automatically optimize and operate the subject peptide manufacturing (i.e., controller and associated machine parts, reactors, etc.). Further, some embodiments incorporate immediate feedback, e.g., before the next reaction reagents that enter into the flow stream headed to the reactor, e.g., resin, and responses into the automated and robotic system which enables the machine learning implemented functionality described herein to intelligently manipulate different continuous variables (e.g., double coupling, double deprotection, temperature, elapsed time, and concentration) for optimal synthesis. Advantageously, embodiments improve peptide synthesis and production in SPSF environments in contrast to continuous flow synthesis systems and environments as detailed next.

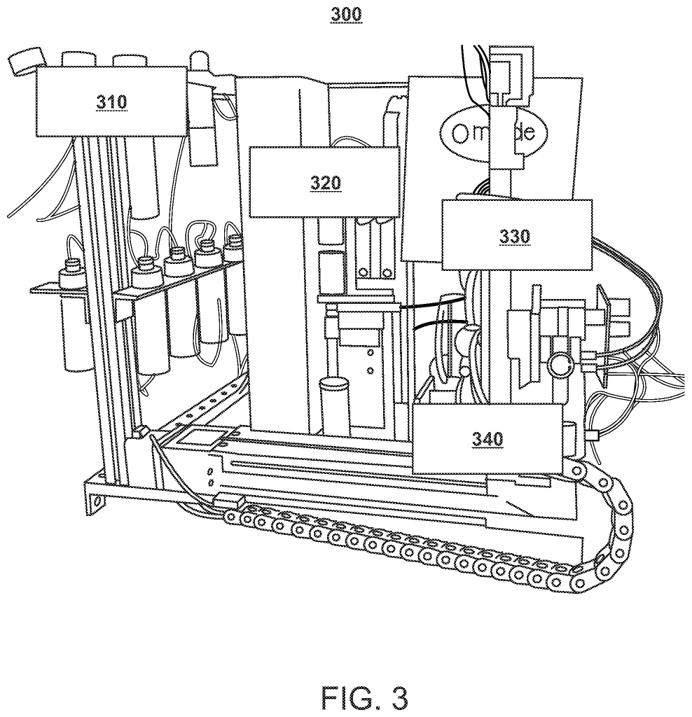

FIG. 3 illustrates an example system 300 in which embodiments, e.g., the method 220, may be integrated. Such an embodiment is integrated into the bench top apparatus 300 that moves the reagents 310 utilizing continuous solid phase peptide synthesis and is capable of carrying out 10 syntheses with high yield at 2 minutes per coupling cycle. The system 300 utilizes fluidic components, such as an automated reactor 320 and reaction vessels 330, to accurately mix and meter reagents. In addition, a robotic arm 340 allows automated synthesis of up to 40 peptides before needing to be reloaded with new reaction vessels. In an embodiment, software developed for the apparatus 300 implements the embodiments described herein, e.g., the method 220, to optimize peptide manufacturing performed by the apparatus 300. In certain other embodiments, system 300 has an integrated UV detector capable of capturing data at the outlet of the reactor 320. This captured data can, in turn, be used by embodiments to optimize peptide manufacturing being performed by the apparatus 300.

Embodiments can be integrated in the apparatus 300 to provide machine learning-driven process condition optimization. In such an embodiment, data can be collected by the apparatus 300, and output predictions provided by an embodiment, can be used to tune operating conditions, e.g., knob settings, valve settings, and the like, on the machine 300, for new peptide synthesis.

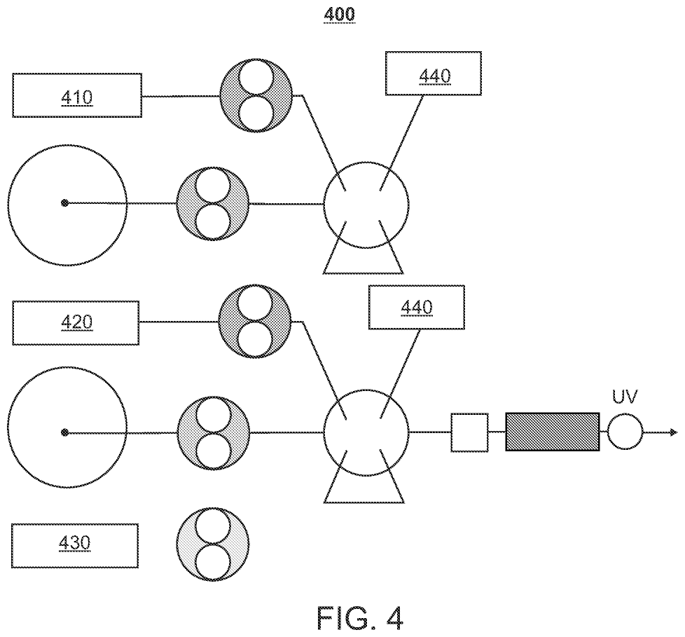

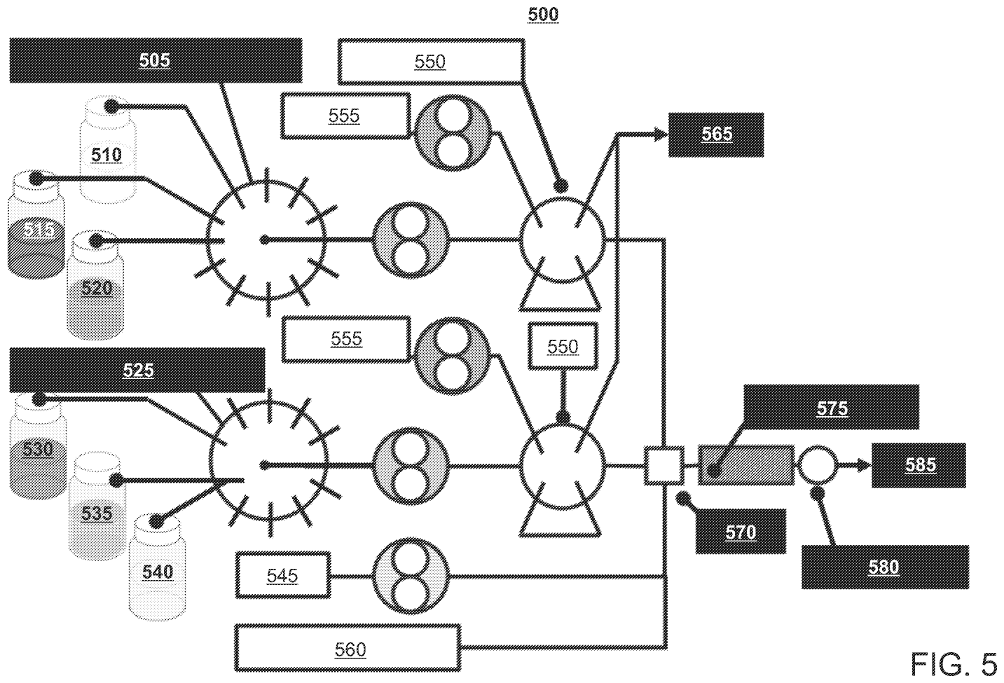

Embodiments can be employed to control any peptide manufacturing process described in International Application No. PCT/US2020/037441. For instance, in some embodiments, the methods and systems for peptide synthesis optimization described herein are implemented in the exemplary systems 400 and 500 of FIGS. 4 and 5, respectively, to perform slug flow processes. The methods and systems described herein can optimize slug flow synthesis implemented by the system 400 and system 500.

In such embodiments, slug flow peptide or protein synthesis can be performed, in which fluid, of one form or another, is transported over the immobilized peptides or proteins 575. In certain embodiments, reagents, e.g., amino acid 410, activator 420, and base 430, are transported throughout the system by slug flow. In the system 400 the valves 440, e.g., 6-port 2 position valves, are configured to load sample loops and inject their contents into a reactor. In certain other embodiments, reagents, e.g., amino acids 510, 515, 520, activators N,N′-diisopropylcarbodiimide (DIC) 530, piperidine (PIP) 535, 2-(1H-Benzotriazole-1-yl)-1,1,3,3-tetramethyluronium hexafluorophosphate (HBTU) 540, and base 545, and washing solvents 555 may be transported by slug flow over the immobilized peptide or protein 575. In other embodiments, amino acids or peptides may be immobilized on a resin 575, e.g., solid support. The resin 575 may be contained within a reaction vessel. In certain embodiments, a plurality of reagent reservoirs, e.g., amino acids 510, 515, 520, activators DIC 530, PIP 535, HBTU 540, and base 545, may be located upstream of and connected to the reactor (FIG. 5).

In some embodiments, a reagent reservoir contains amino acids or peptides, e.g., pre-activated amino acids or peptides and/or amino acids or peptides that are not fully activated. In certain embodiments, a reagent reservoir contains an amino acid activating agent, e.g., an alkaline liquid 545, a carbodiimide 530, and/or a uronium activating agent 540, capable of completing the activation of the amino acids 510, 515, 520.

In still other embodiments, a reagent reservoir contains a deprotection agent, e.g., piperidine 535 or trifluoroacetic acid. A reagent reservoir may contain a solvent 555, e.g., dimethyl formamide (DMF) that may be used in a reagent removal step. While single reservoirs have been illustrated in FIG. 5 for simplicity, it should be understood that in embodiments, where single reservoirs are illustrated, multiple reservoirs, e.g., each containing different types of amino acids, different types of deprotection agents, etc., can be used in place of the single reservoir.

In the system 400 of FIG. 4 and the system 500 of FIG. 5, a computing device integrated into the system (not shown) controls the various input parameters to the system. In such an implementation, a machine learning model based method, e.g., the method 220, is used to identify optimized values for control parameters for the systems 400 and 500 and control operations of the systems 400 and 500.

For instance, embodiments can be used in the system 500 to control mixing time, washout time, separation and reaction time for slug flow techniques so as to enable rapid slug formation with real-time process analytics that allows a robust, unattended, high throughput operation for peptide and protein synthesis. In an example implementation, the slugs are formed in mixer 570 through the combination of reagents (amino acids 510, 515, 520, activators DIC 530, PIP 535, HBTU 540, and base 545) prepared in sample loops 550 that deliver wash solvents 555. In such embodiments, the choice of reagents is selected based on the amino acid selector 505 and the activator selector 525. To isolate the slugs from each other, separation media 560 can be added at the beginning and end of the reagent load step, encapsulating the slugs within the reagent delivery system. In other embodiments, the separation media 560 can be an inert gas or oil, to limit dispersion and slug mixing within the system. In such embodiments, a slug's internal mixing occurs based on the material interface. In other embodiments, the viscous drag between the moving slug and tube wall causes relative motion of the fluid which is recirculated by the interface resulting in robust internal circulation. In such embodiments, the robust mixing results in a homogenous slug that can be delivered to a reactor containing solid support resin 575 preventing concentration gradients from forming. In still other embodiments, the implementation of conductivity meter 580 into the system can enable the slug flow technology to reduce the amount of waste 565 and 585 generated by the method.

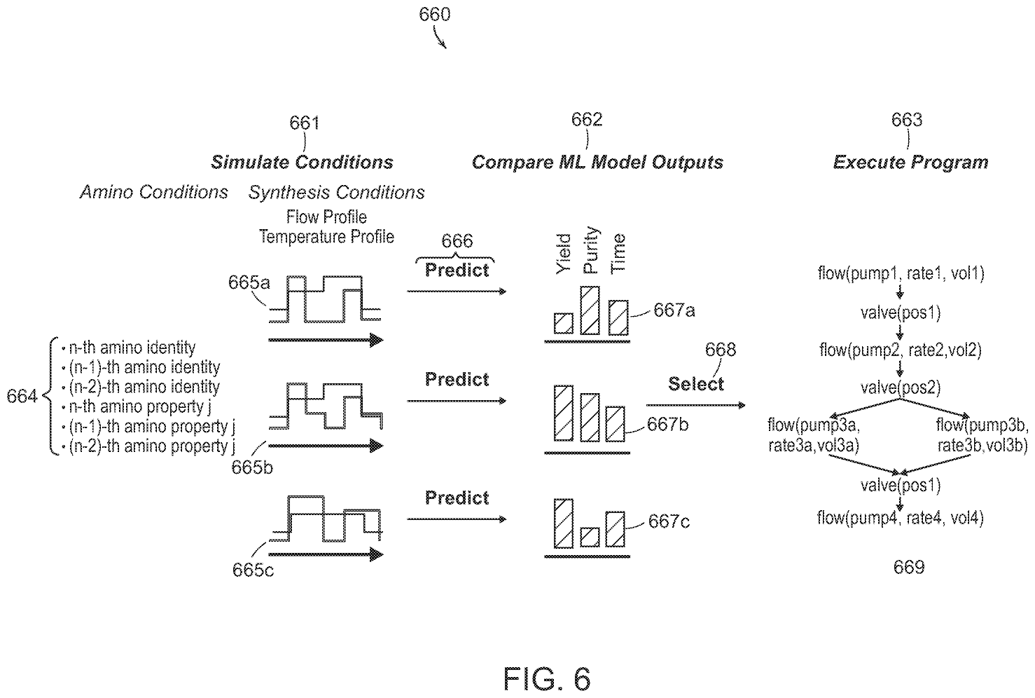

FIG. 6 is a graphical illustration of a method 660 that uses machine learning to synthesize a peptide. The method 660 includes three phases: (i) condition simulation 661, (ii) machine learning output comparison 662, and (iii) program execution 663, i.e., peptide synthesis.

The condition simulation 661 phase begins with receiving the amino conditions 664. In the conditions 664, the notation “n-th” amino acid in the polypeptide chain refers to the current one, where n=1, 2, 3, . . . . The (n−1)-th amino acid is the previous one. The (n−2)-th amino acid is the one before that. Identity refers to the amino acid residue, e.g., Glycine, Alanine, etc. In the method 660 there is a list of amino acid properties which are indexed by the letter j. The properties include residue molecular weight, hydrophobicity, and size, amongst other. The properties are fixed features in the machine learning model for the current residue. The conditions (e.g., profiles 665a-c) are adjustable.

The amino conditions 664 are used in conjunction with candidate synthesis conditions 665a, 665b, 665c to predict 666 synthesis quality. In the example of FIG. 6, each of the candidate synthesis conditions 665a, 665b, and 665c, include a respective flow rate profile and temperature profile. To continue, the various candidate synthesis conditions 665a, 665b, and 665c are concatenated with the conditions 664, e.g., to create a vector, and this concatenated data is provided to a machine learning model that is trained to predict 666 the quality of peptide synthesis carried out using the candidate synthesis conditions 665a, 665b, and 665c concatenated with the conditions 664. In an example, each condition 665a-c is concatenated with the conditions 664 to form respective vectors (not shown) that are used in the machine learning engine to predict 666 the synthesis qualities 667a-c.

The method 660 then moves to the comparison phase 662 where the outputs 667a, 667b, and 667c of the machine learning model are compared. In the example method 660 of FIG. 6, the machine learning model outputs 667a, 667b, and 667c each include an indication of predicted yield, purity, and time (production time or elapsed time). Based on the comparison 662 of the machine learning model outputs 667a, 667b, and 667c, a set of synthesis conditions from among the candidate synthesis conditions 665a, 665b, and 665c is selected 668. In embodiments, the outputs of the machine learning model, e.g., the predicted yield, purity, and production time, may be weighted according to user preference for the comparison 662 and selection 668. For instance, a user assigned weight may be assigned to each component, e.g., yield, purity, and production time, of the machine learning model outputs 667a, 667b, and 667c to determine a user desired ranking of the candidate synthesis conditions 665a, 665b, and 665c. An embodiment could weigh them by assigning weight=1 to yield and weight=0 to the others, for example. A hybrid can also be used, for instance, where the production is under some threshold time, while maximizing yield among the remaining allowed conditions.

The method 660 next executes 663 the peptide synthesis using the selected 668 synthesis conditions. Executing 663 the synthesis includes executing a sequence 669 of operations. The sequence 669 is but one example sequence that may be utilized in embodiments. In embodiments, sequences may include actuating the appropriate pumps, valves, heaters, etc. sequentially and in parallel to achieve the selected 668 synthesis conditions.

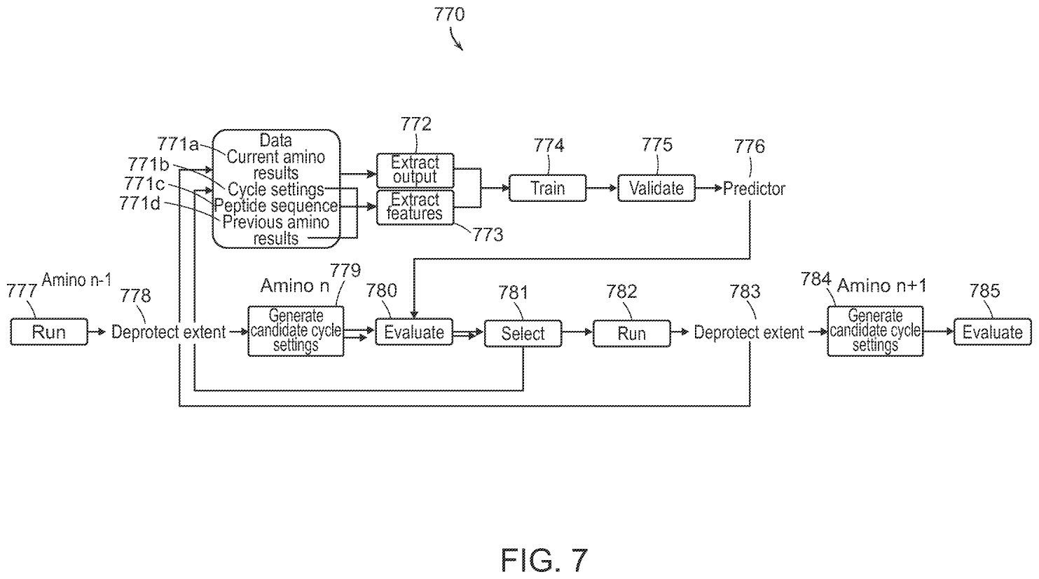

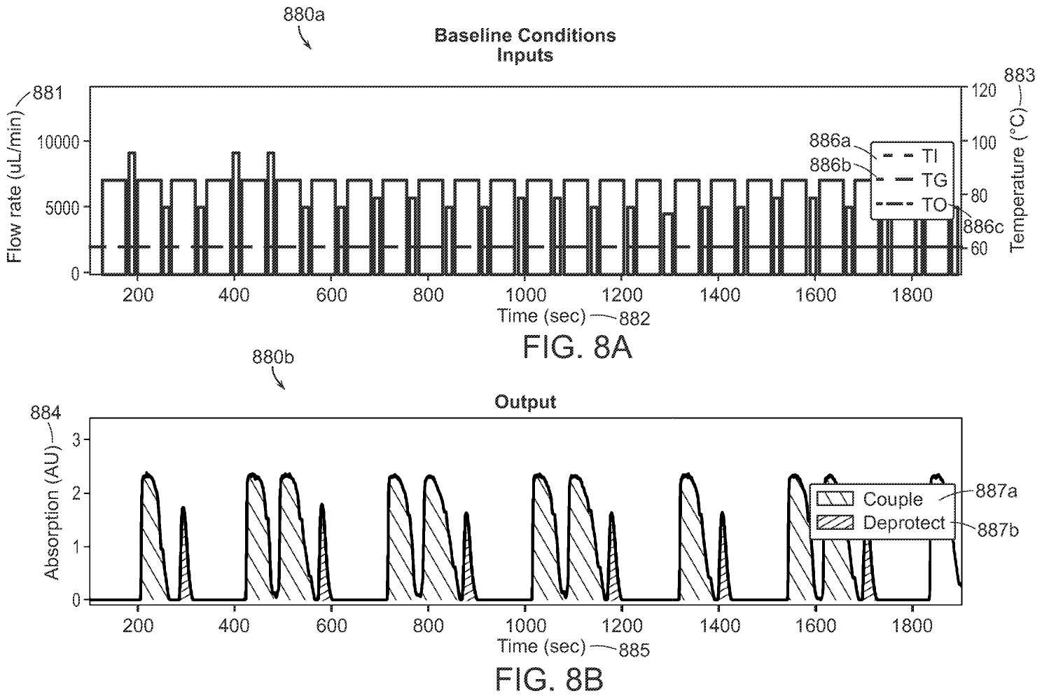

FIG. 7 is a flowchart of a method 770 for training a machine learning model and controlling peptide production using the trained machine learning model according to an embodiment. The method 770 begins with the data (generally referred to as 771) that includes the current amino results 771a, cycle settings 771b, peptide sequence 771c, and previous amino results 771d. The method 770 extracts the output 772 from the current amino results 771a and extracts features 773 from the cycle settings 771b, peptide sequence 771c, and previous amino results 771d data. According to an embodiment, extracting the output 772 includes determining the baseline conditions. For instance, extracting the output may include generating the plots 880a and 880b described hereinbelow in relation to FIGS. 8A and 8B and calculating the extent of reaction. Such functionality may include splitting a time trace of UV absorbance into a per-amino acid, per-couple/deprotect buckets, dividing by a flow rate, and multiplying by a constant, and integrating. This determines the yield, which is directly proportional to extent of reaction. Extracting the outputs 772 can also include calculating the time spent for each amino acid (because the system may have to wait to reach the desired temperature). According to an embodiment, extracting the features 773 entails copying conditions, such as flow profile and temperature profile, amongst others, fed to the machine.

The extracted output 772 and extracted features 773 are then used to train 774 the machine learning model. Embodiments of the method 770 may utilize any training 774 technique known to those of skill in the art for the machine learning model(s) that are employed. One such embodiment utilizes a gradient descent technique to optimize a loss function, which is the squared difference between the predicted and actual output.

To continue, the trained 774 machine learning model is validated 775 which results in the machine learning model, i.e., peptide synthesis quality predictor 776. Embodiments of the method 770 may utilize any machine learning validation 775 technique known to those of skill in the art. In an embodiment of the method 770 the available data (each sample's inputs and output) are randomly split into a training (70%) set and validation (30%) set. Model training 774 is performed on the training data (70%). Model performance is evaluated 775 on the validation data, which the model has never seen before. The performance on the validation data is reported.

During peptide synthesis carried out by the method 770, the previous amino 777 and indication of the extent of deprotection 778 from the previous amino is used as input to generate 779 candidate cycle settings. The generated 779 candidate settings are evaluated 780 using the predictor 776 and a given set of candidate settings are selected 781. The selected 781 settings are used to run 782 the peptide synthesizer to add the amino acid to the polypeptide chain. An indication of the deprotection extent 783 is then used to restart the process at step 784 to generate candidate cycle settings and evaluate the settings 785 for the next amino acid.

In the method 770, the deprotection extent 783 data is also added to the data 771d for training future machine learning models.

FIG. 8A is a plot 880a showing baseline input for a method for peptide production. The plot 880a shows baseline flow rate 881 versus time 882 and the temperature 883 associated with the flow rate 881. In other words, the plot 880a shows flowrate for a peptide synthesis process that is not optimized using the functionality described herein. In the plot 880a, the series 886a, 886b, and 886c show temperature profiles for three temperature control units inside and adjacent to the reactor. In the plot 880, the series 886a is TI=inlet temperature; the series 886b is TG=gripper (closest to the reactor) temperature; and the series 886c is TO=outlet temperature. The plot 880b shows the resulting absorption 884 versus time 885 that results when peptide production is carried out using the non-optimized flow rate 881 shown in the plot 880a in FIG. 8A. In the plot 880b, the shading shows the absorption 884 throughout the process during the couple 887a and deprotect 887b stages of the peptide production.

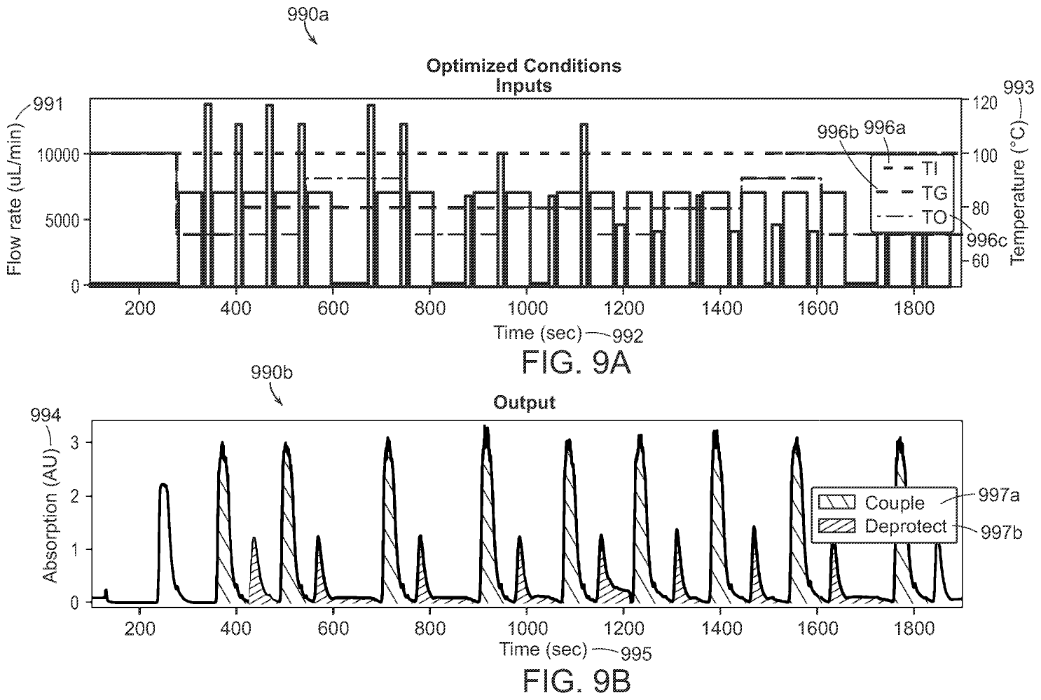

FIG. 9A is a plot 990a showing the results of optimizing the baseline flow rate 881 of FIG. 8A. The plot 990a shows optimized flow rate 991 versus time 992 and the temperature 993 associated with the flow rate profile 991. In the plot 990a, the series 996a, 996b, and 996c show corresponding temperature profiles as 886a, 886b, and 886c.

FIG. 9B is a plot 990b showing the output (absorption 994) versus time 995 that results when peptide production is carried out using the optimized flow rate 991 of FIG. 9A. In the plot 990b, the shading shows the absorption 994 throughout the production process during the couple 997a and deprotect 997b stages of the peptide production method.

According to an embodiment, the machine learning model is configured to receive predictor inputs. These inputs may include the parameters of the peptide synthesis machine and the peptide sequence and derived features. Example inputs include, amongst others, (i) current amino acid identity, (ii) previous N amino acids (already coupled to the chain) identities, (iii) physical and chemical properties of amino acids, (iv) flow rates (e.g., flow rate profiles) for load, couple, deprotect, wash stages (elapsed time per step is implicitly calculated from flow rates), and (v) temperatures for couple, deprotect, wash stages.

Further, an example embodiment configures, e.g., trains, the machine learning model to provide an output that indicates predicted yield quality. Outputs may include quantities to maximize such as yield and purity and quantities to minimize such as aggregation propensity, side product formation, and purification difficulty. Example outputs include integrated area under a UV trace at 200-300 nm and integrated area under a UV trace at 200-300 nm, where the UV trace values have been normalized with respect to the instantaneous flow rate past the UV detector.

An embodiment uses a different machine learning predictor 776 for each output, e.g., indication of quantity to minimize, or quantity to maximize. These predictors can be trained on a set of available synthesis data and validated on a held-out portion (portion not used in the training). Models such as logistic regression and random forests can be used to predict categorical outputs such as un/acceptable yield from basic protocol parameters and presence of amino acids in the sequence. Further, more complex models such as deep neural nets can be used to predict continuous outputs such as a combined “synthesizability” score from a more complete set of protocol parameters and raw representation of peptide composition. Model training 774 can utilize GPU and cloud computing resources running machine learning tools such as XGBoost and PyTorch. Models can also be retrained and revalidated as new peptides are synthesized and data, e.g., UV trace, is collected.

An embodiment trains 774 the machine learning model to provide the indicator of output quality given the input of operating conditions. An embodiment trains the machine learning model using a training dataset that includes peptides run on the synthesizer apparatus (until the date the data was gathered). According to an embodiment, each synthesis from which the training data was obtained generates as many samples as couplings, of the inputs and outputs of the machine learning model. An embodiment may also take additional peptides run on a synthesizer with randomized conditions for the predictor 776 inputs.

Embodiments may utilize a peptide quality predictor 776 that is a supervised machine learning model used for regression. One such embodiment, uses a LASSO model and a random forest regressor model. Embodiments can use the aforementioned predictor inputs, predictor outputs, and model with a training 774 and validation 775 procedure, as is known in the art, to generate a machine learning model to predict production quality of a peptide production system.

An example embodiment utilizes a machine learning model predictor 776 as part of an optimization system. In such an implementation, for each amino acid that will be part of a chain, an ensemble of predictor inputs is generated, either randomly (between bounds) or exhaustively (Cartesian product of selected levels between the bounds). In turn, the machine learning predictor 776 is run on each sample to determine predicted quality of each set of predictor inputs. The operating conditions (values and settings therefor) are then selected from the sample with optimal predicted value, either the highest yield or the shortest time that gave a yield above some threshold.

After the operating conditions are selected, an embodiment synthesizes the peptide with these conditions (values and corresponding settings). After synthesizing the peptide, values for the input and output conditions resulting from synthesizing the peptide are collected and used to further train the model.

An embodiment predicts production quality for each amino acid in a chain before starting synthesis of that peptide. Another embodiment implements just-in-time predictions where a prediction is determined and conditions controlled on a per-amino basis, immediately before each amino acid is synthesized during operation.

In an embodiment, the output of the machine learning engine output corresponds to a quantity that can be calculated, measured, or otherwise determined from operating the peptide manufacturing system, e.g., extent of reaction, time taken, etc.). Given a desired amino acid identity to be synthesized, and property and process condition features output of the machine learning engine, an embodiment provides an “end-to-end” ML system that directly relates them.

The machine learning-based optimization described herein supports and enhances the operation of the solid phase slug flow peptide synthesizer described in International Application No. PCT/US2020/037441. Embodiments provide a scalable, i.e., functionality that is able to incorporate a lot of data including all past runs so as to continuously improve, mechanism for improving peptide synthesis. Moreover, embodiments improve the purity of peptide synthesis and can thus, reduce, or eliminate the need for purification.

The machine learning functionality described herein enables real-time proposal, outcome prediction, and selection of candidate process steps, something no other technology can currently perform. Conducting real-time process step selection facilitates the orchestration of asynchronous production events. Further, advanced control techniques can employ industrial process controllers to provide reliable and repeatable timing and coordination.

Further, it is noted that while embodiments are described as being used to control peptide production, embodiments are not so limited and can be applied to any system that uses “building block” chemistry (“-tides”).

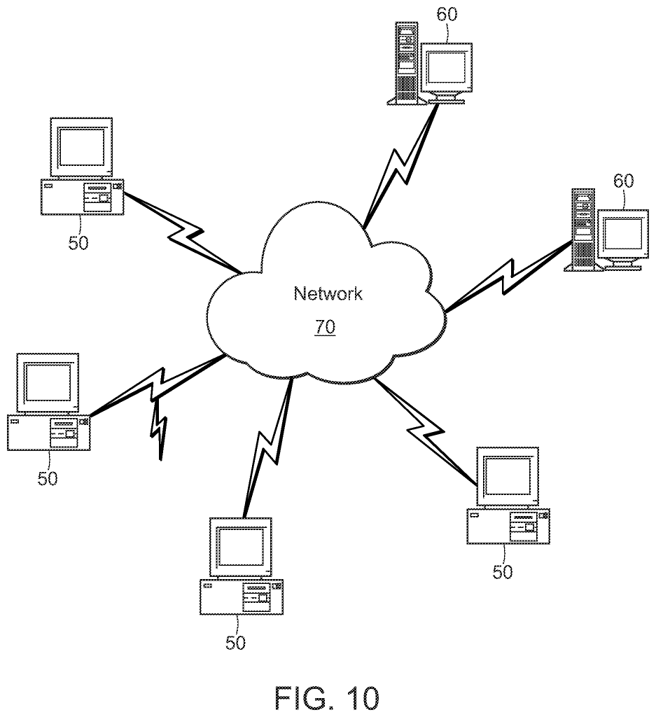

FIG. 10 illustrates a computer network or similar digital processing environment in which embodiments of the present disclosure may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

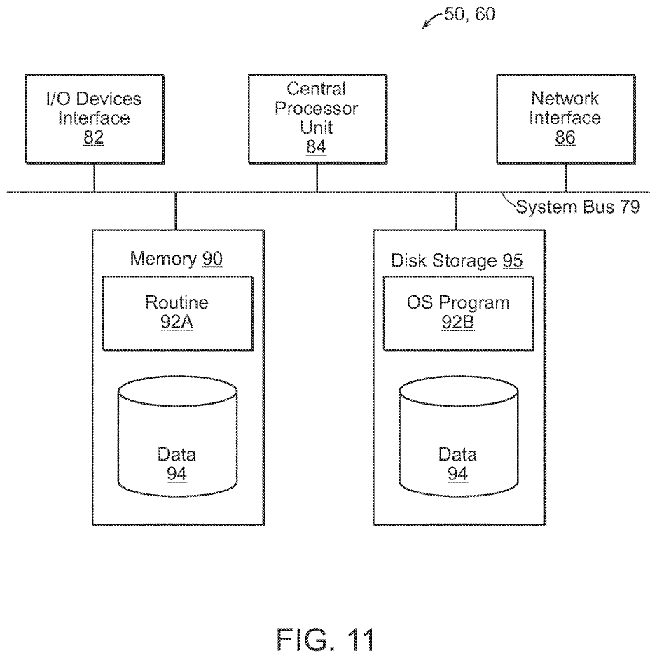

FIG. 11 is a diagram of an example internal structure of a computer (e.g., client processor/device 50 or server computers 60) in the computer system of FIG. 10. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 10). Memory 90 provides volatile storage for computer software instructions 92A (of Methods 220, 660, 770) and data 94 used to implement an embodiment of the present disclosure. Disk storage 95 provides non-volatile storage for computer software instructions 92B (of Methods 220, 660, 770) and data 94 used to implement an embodiment of the present disclosure. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for an embodiment. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

All patents, published applications, and references mentioned herein are hereby incorporated by reference in their entirety as if each individual patent, published application, or reference was specifically and individually indicated to be incorporated by reference. In case of conflict, the present application, including any definitions herein, will control.

While specific aspects and embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

While example embodiments have been particularly shown and described, it will be understood by those skilled in the art that various changes in form and details may be made therein without departing from the scope of the embodiments encompassed by the appended claims.

What is claimed is:

1. A method for controlling peptide production, the method comprising:
providing a manufacturing process that synthesizes peptides using solid phase slug flow;
automating the manufacturing process through use of a machine learning engine, wherein (i) automating the manufacturing process comprises selecting values for operating conditions for the manufacturing process and (ii) a given operating condition is flow rate profile; and
generating an indication of the selected values for the operating conditions.

2. The method of claim 1 wherein selecting values for operating conditions for the manufacturing process comprises a processor:
determining candidate values for the operating conditions for a plurality of peptide production scenarios;
predicting quality of each of the plurality of peptide production scenarios by using the determined candidate values in the machine learning engine, wherein the machine learning engine is configured to output an indication of predicted production quality given the candidate values for the operating conditions; and
selecting a peptide production scenario from among the plurality of peptide production scenarios based upon the indication of predicted production quality for each of the plurality of peptide production scenarios, wherein candidate values for the operating conditions of the selected peptide production scenario correspond to the selected values for the operating conditions for the manufacturing process.

3. The method of claim 2 wherein determining the candidate values for the operating conditions comprises at least one of:
randomly generating candidate values between an upper bound and a lower bound for an operating condition; and
generating candidate values in set increments between an upper bound and a lower bound for an operating condition.

4. The method of claim 2 wherein the indication of predicted production quality indicates at least one of: production yield, peptide purity, and production time.

5. The method of claim 4 wherein the indication of production yield corresponds to an integral of ultraviolet (UV) absorbance trace over time of flow-through reaction products.

6. The method of claim 4 wherein the indication of production yield corresponds to an extent of reaction determined by dividing a measured UV trace by an instantaneous flow rate and multiplying by a constant.

7. The method of claim 1 wherein the operating conditions further include at least one of: current amino acid position, current amino acid identity, previous amino acid, physical properties of an amino acid, chemical properties of an amino acid, oscillation frequency, and temperature.

8. The method of claim 7 wherein the temperature indicates a temperature for each of a plurality of stages of the manufacturing process.

9. The method of claim 1 wherein the flow rate profile indicates flow rates for each of a plurality of stages of the manufacturing process.

10. The method of claim 9, wherein the plurality of stages include: load, couple, capping, deprotect, and wash.

11. The method of claim 1 further comprising:
controlling the manufacturing process in accordance with the generated indication of the selected values for the operating conditions.

12. The method of claim 1 wherein the steps of providing, automating and generating are computer implemented, and
the generated indication of the selected values for the operating conditions enables computer automated control of the manufacturing process.

13. A system for controlling a manufacturing process that synthesizes peptides using solid phase slug flow, the system comprising:
a processor; and
a memory with computer code instructions stored thereon, the processor and the memory, with the computer code instructions, being configured to cause the system to:
automate the manufacturing process through use of a machine learning engine, wherein (i) automating the manufacturing process comprises selecting values for operating conditions for the manufacturing process and (ii) a given operating condition is flow rate profile; and
generate an indication of the selected values for the operating conditions.

14. The system of claim 13 wherein, in selecting values for operating conditions for the manufacturing process, the processor and the memory, with computer code instructions, are further configured to cause the system to:
determine candidate values for the operating conditions for a plurality of peptide production scenarios;
predict quality of each of the plurality of peptide production scenarios by using the determined candidate values in the machine learning engine, wherein the machine learning engine is configured to output an indication of predicted production quality given the candidate values for the operating conditions; and
select a peptide production scenario from among the plurality of peptide production scenarios based upon the indication of predicted production quality for each of the plurality of peptide production scenarios, wherein candidate values for the operating conditions of the selected peptide production scenario correspond to the selected values for the operating conditions for the manufacturing process.

15. The system of claim 14 wherein, in determining the candidate values for the operating conditions, the processor and the memory, with the computer code instructions, are further configured to cause the system to perform at least one of:
randomly generating candidate values between an upper bound and a lower bound for an operating condition; and
generating candidate values in set increments between an upper bound and a lower bound for an operating condition.

16. The system of claim 14 wherein the indication of predicted production quality indicates at least one of: production yield, peptide purity, and production time.

17. The system of claim 16 wherein the indication of production yield corresponds to an integral of ultraviolet (UV) absorbance trace over time of flow-through reaction products.

18. The system of claim 16 wherein the indication of production yield corresponds to an extent of reaction determined by dividing a measured UV trace by an instantaneous flow rate and multiplying by a constant.

19. The system of claim 13 wherein the operating conditions further include at least one of: current amino acid position, current amino acid identity, previous amino acid, physical properties of an amino acid, chemical properties of an amino acid, oscillation frequency, and temperature.

20. The system of claim 19 wherein the temperature indicates a temperature for each of a plurality of stages of the manufacturing process.

21. The system of claim 13 wherein the flow rate profile indicates flow rates for each of a plurality of stages of the manufacturing process.

22. The system of claim 21, wherein the plurality of stages include: load, couple, capping, deprotect, and wash.

23. The system of claim 13 wherein the processor and the memory, with the computer code instructions, are further configured to cause the system to:
control the manufacturing process in accordance with the generated indication of the selected values for the operating conditions.

24. The system of claim 13 wherein the generated indication of the selected values for the operating conditions enables control of the manufacturing process.

25. A computer program product for controlling a manufacturing process that synthesizes peptides using solid phase slug flow, the computer program product comprising:
one or more non-transitory computer-readable storage devices and program instructions stored on at least one of the one or more storage devices, the program instructions, when loaded and executed by a processor, cause an apparatus associated with the processor to:
automate the manufacturing process through use of a machine learning engine, wherein (i) automating the manufacturing process comprises selecting values for operating conditions for the manufacturing process and (ii) a given operating condition is flow rate profile; and
generate an indication of the selected values for the operating conditions.
